(19) United States
(12) Patent Application Publication
(10) Pub. No.: US 2006/0270685 A1
(43) Pub. Date: Nov. 30, 2006

(54) Anhydrous ziprasidone mesylate and a process for its preparation

(76) Inventors: Judith Aronhime, Rehovot (IL); Marioara Mendelovici, Rehovot (IL); Sigalit Levi, Modi'in (IL)

Correspondence Address: KENYON & KENYON LLP, ONE BROADWAY, NEW YORK, NY, 10004, US

(21) Appl. No.: 11/376,551

(22) Filed: Mar. 14, 2006

Related U.S. Application Data
(60) Provisional application No. 60/687,946, filed on Jun. 6, 2005.
(60) Provisional application No. 60/661,687, filed on Mar. 14, 2005.
(60) Provisional application No. 60/689,701, filed on Jun. 9, 2005.
(60) Provisional application No. 60/705,762, filed on Aug. 4, 2005.
(60) Provisional application No. 60/762,349, filed on Jan. 25, 2006.
(60) Provisional application No. 60/762,695, filed on Jan. 26, 2006.

(51) Int. Cl. A61K 31/496; C07D 417/14

(52) U.S. Cl. 514/254.06; 544/368

(57) ABSTRACT

Provided is anhydrous ziprasidone mesylate polymorph and processes for its preparation.

### RELATED APPLICATIONS

The present application claims the benefit of the following U.S. Provisional Patent Application Nos.: 60/687946 filed Jun. 6, 2005, Ser. No. 60/661687 filed 14 Mar. 2005, 60/689701 filed 9 Jun. 2005, 60/705762 filed 4 Aug. 2005, 60/762349 filed 25 Jan. 2006 and 60/762695 filed 26 Jan. 2006. The contents of which are incorporated herein by reference.

### FIELD OF INVENTION

The present invention is directed to anhydrous ziprasidone mesylate having a tabular and equant morphology, as well as processes for its preparation.

### BACKGROUND OF THE INVENTION

Ziprasidone is an antipsychotic agent and is therefore useful for treating various disorders including schizophrenia, anxiety and migraine pain. Ziprasidone has the following structure: 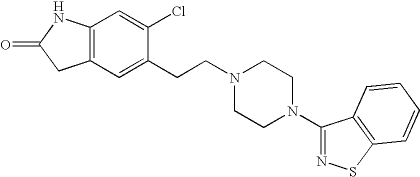
Ziprasidone has been marketed under the name GEODON as an oral capsule and as an injectable drug. GEODON capsules contain the monohydrate hydrochloride salt of ziprasidone, and come in 20, 40, 60 and 80 mg dosage forms. GEODON for injection contains a lyophilized form of ziprasidone mesylate trihydrate, and contains 20 mg base equivalent of ziprasidone.

The present invention relates to the solid state physical properties of ziprasidone mesylate. These properties may be influenced by controlling the conditions under which ziprasidone mesylate is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate. Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and may be used to distinguish some forms from others. A particular form may also give rise to distinct spectroscopic properties that may be detectable by powder X-ray crystallography, solid state C NMR spectrometry and infrared spectrometry.

The preparation of ziprasidone base is disclosed in U.S. Pat. No. 4,831,031 (example 16). Preparation of ziprasidone base is also disclosed in U.S. Pat. No. 5,312,925. U.S. Pat. No. 6,245,765 discloses dihydrate crystalline salts of ziprasidone mesylate and their use as dopamine antagonists. Also disclosed is anhydrous ziprasidone mesylate characterized by X-ray powder diffraction peaks at 19.8, 21.6, 23.0, 24.7 and 26.5±0.2 degrees two-theta. Anhydrous ziprasidone mesylate may be further characterized by X-ray powder diffraction peaks at 12.9, 16.4, 17.7, 22.7 and 23.9±0.2 degrees two-theta. U.S. Pat. No. 6,110,918 discloses four known ziprasidone mesylate crystalline forms. Each crystal form may be characterized by a distinct X-ray powder diffraction pattern and a distinct crystal shape that can be observed by photomicrograph. U.S. Pat. No. 6,110,918 also reports that the ziprasidone mesylate dihydrate lath crystals and dihydrate needle crystals are relatively long and thin in contrast to the prism crystals of ziprasidone mesylate trihydrate. The anhydrous ziprasidone mesylate is said to be hygroscopic when exposed to air (humidity). In an aqueous medium at ambient temperature, ziprasidone mesylate trihydrate is reported to be the most thermodynamically stable form of the four crystalline forms of ziprasidone mesylate. U.S. Pat. No. 6,399,777 discloses the preparation of ziprasidone mesylate anhydrous forms by slurrying ziprasidone base in isopropyl alcohol. The anhydrous ziprasidone mesylate is obtained by the process in U.S. Pat. No. 6,399,777. Example 3 in U.S. Pat. No. 6,245,765 teaches the preparation of anhydrous ziprasidone mesylate crystals having a lath morphology by adding methanesulfonic acid to the slurry of Ziprasidone base in isopropyl alcohol at 50° C.

Repeating the procedure of U.S.'765 yielded anhydrous ziprasidone mesylate having a high residual solvent content (measured by GC head space method) of 0.8 to 1% isopropyl alcohol.

Publication No. US 2004/0194338 discloses a multitude of dispersions containing amorphous drugs and polymers, prepared by spray drying.

The discovery of new forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for additional forms of ziprasidone mesylate and/or processes for their preparation.

### SUMMARY OF THE INVENTION

One aspect of the present invention is anhydrous ziprasidone mesylate having a tabular and equant morphology.

Another aspect of the present invention is a non-hygroscopic anhydrous ziprasidone mesylate.

Yet another aspect is a non-hygroscopic anhydrous ziprasidone mesylate having a tabular and equant morphology.

A further aspect of the present invention is anhydrous ziprasidone mesylate with a tabular and equant morphology, having a low residual solvent content.

One aspect is a new process for the preparation of anhydrous ziprasidone mesylate having a tabular and equant morphology, comprising desolvation of solvated or hydrated forms of ziprasidone mesylate, and ziprasidone mesylate anhydrous having a tabular and equant morphology, prepared by this process.

Another aspect of the present invention is a process for preparing anhydrous ziprasidone mesylate having a tabular and equant morphology, comprising, heating a crystalline form of ziprasidone mesylate. This crystalline form may be any one of: Form I, Form II, Form III, Form IV, Form V, Form VI, Form VII, Form VIII, Form IX, From X, Form XIII and amorphous form. The present invention also comprises the anhydrous ziprasidone mesylate having a tabular and equant morphology obtained by the above heating processes.

Yet another aspect of the present invention is a process for preparing anhydrous ziprasidone mesylate having a tabular and equant morphology comprising combining ziprasidone base with methane-sulfonic acid in the presence of a solvent selected from the group consisting of a C₂ to C₈ ketone, a C₃-C₄ alcohol (preferably the alcohol is other than isopropanol) and mixtures thereof with water, an amide (preferably dimethylformamide), chlorinated C₁ to C₄ alkanes (preferably chlorinated C₁ to C₂ alkane, such as dichloromethane), a C₂ to C₈ ether, a C₂ to C₈ alkyl ester, acetonitrile, 2-ethoxy ethanol, dioxane and a mixture of THF and water, to obtain a reaction mixture; and maintaining the reaction mixture for a time sufficient to obtain anhydrous ziprasidone mesylate having a tabular and equant morphology. The present invention also comprises the anhydrous ziprasidone mesylate having a tabular and equant morphology obtained by this process.

A further aspect of the present invention is a process for preparing anhydrous ziprasidone mesylate having low residual solvent content comprising adding methanesulfonic acid to a slurry of ziprasidone base in dry isopropanol, to obtain a reaction mixture, and stirring the reaction mixture to obtain anhydrous ziprasidone mesylate.

One aspect of the present invention is pharmaceutical formulations (especially solid pharmaceutical formulations (e.g. tablets, capsules, powder, granules, suppositories, suspensions, etc.) comprising anhydrous ziprasidone mesylate having a tabular and equant morphology and a pharmaceutically acceptable carrier.

Another aspect of the present invention is pharmaceutical formulations comprising anhydrous ziprasidone mesylate with a tabular and equant morphology, having a low residual solvent content, and a pharmaceutically acceptable carrier.

Another aspect of this invention is a solid pharmaceutical formulation containing a lyophilizate of anhydrous ziprasidone mesylate that has a tabular and equant morphology.

Yet another aspect of the present invention is the use of ziprasidone mesylate having a tabular and equant morphology for the preparation of solid pharmaceutical formulation.

Another aspect of the present invention is the use of non-hygroscopic anhydrous ziprasidone mesylate for the preparation of solid pharmaceutical formulation.

A further aspect of the present invention is the preparation of pharmaceutical formulations containing ziprasidone mesylate by lyophilizing anhydrous ziprasidone mesylate having a tabular and equant morphology, or by lyophilizing non-hygroscopic anhydrous ziprasidone mesylate.

A further aspect of the present invention is a solid pharmaceutical formulation, comprising nano-particles of crystalline anhydrous ziprasidone mesylate, that has a tabular and equant morphology, and the process for its preparation.

A further aspect of the present invention is the use of anhydrous ziprasidone mesylate as having tabular and equant morphology, or non-hygroscopic anhydrous ziprasidone mesylate, or anhydrous ziprasidone mesylate having low residual solvent content for the manufacture of a pharmaceutical formulation.

### BRIEF DESCRIPTION OF THE DRAWINGS

FIG. 1: A photomicrograph of Ziprasidone mesylate anhydrous transformed from form I.

FIG. 2: A photomicrograph of anhydrous ziprasidone mesylate prepared by slurrying form I example 4 (in acetone).

FIG. 3: A photomicrograph of anhydrous ziprasidone mesylate prepared by slurrying form I example 4 (in dry isopropanol).

FIG. 4: A photomicrograph of anhydrous ziprasidone mesylate prepared by example 3 (in n-propanol)

FIG. 5: A photomicrograph of anhydrous ziprasidone mesylate prepared by example 3 (in n-butanol)

FIG. 6: A photomicrograph of anhydrous ziprasidone mesylate prepared by example 3 (in tetrahydrofuran)

FIG. 7: A photomicrograph of anhydrous ziprasidone mesylate prepared by example 3 (in ethyl acetate)

### DETAILED DESCRIPTION OF THE INVENTION

As used herein, the term “slurry” refers to a heterogeneous mixture.

As used herein, the term “anhydrous” refers to a crystalline form having water content of not more than about 0.6% by weight. As used herein, the term “non-hygroscopic” refers to a compound that absorbs no more than about 0.2% of water, preferably not more than 0.1% of water and more preferably not more than 0.05% of water at about 80% humidity at a temperature of 25° C. for 24 hours. The definition of this term can be found in: “Pharmeuropa Vol. 4, No. 3, September 1992”.

As used herein, the term “ambient temperature” is meant to indicate a temperature of about 15° C. to about 32° C., preferably about 18 to about 25° C.

As used herein, the term “tabular” refers to flat particles of similar length and width, but possessing greater thickness than flakes. As used herein, the term “equant” refers to particles of similar length, width, and thickness. A base for these terms can be found in: “Physical characterization of pharmaceutical solids” edited by Harry G. Brittain, chapter 5: Particle morphology”. Moreover, tabular and equant morphologies are very different from the lath, needle and prism morphologies.

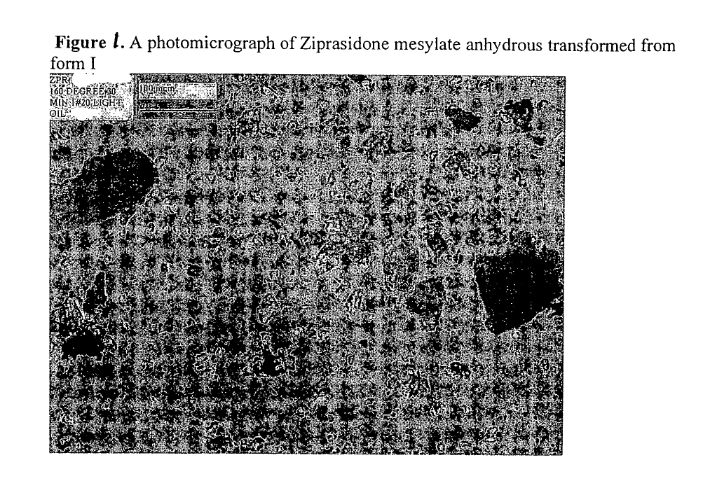

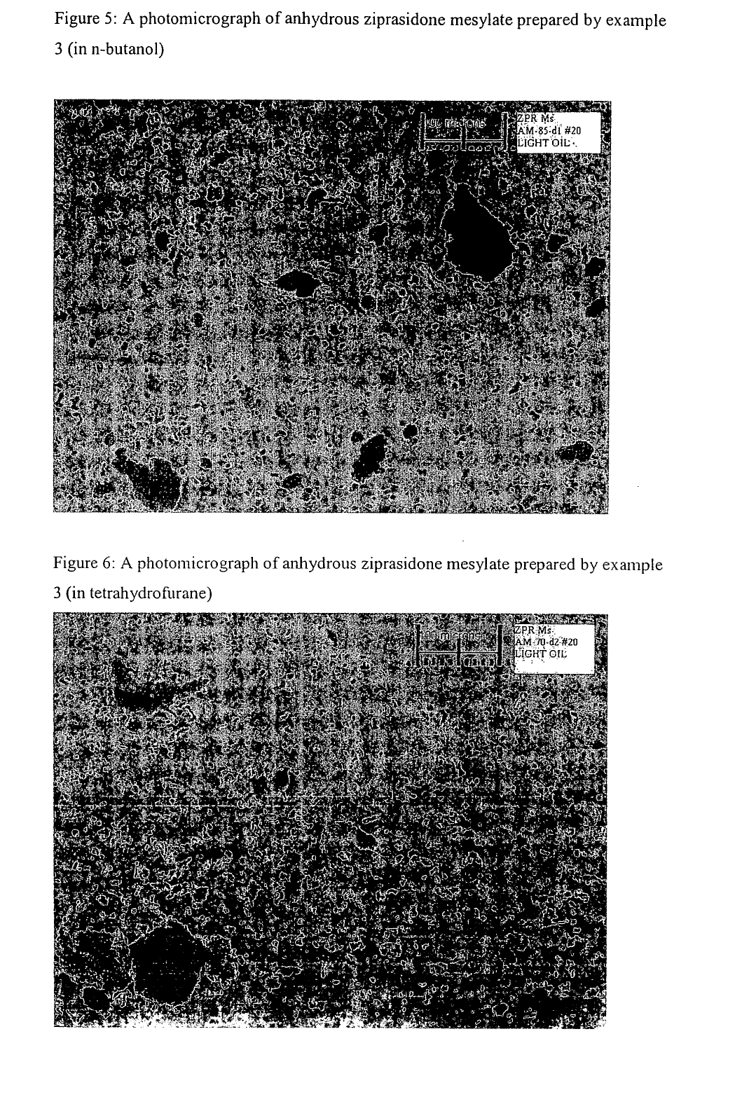

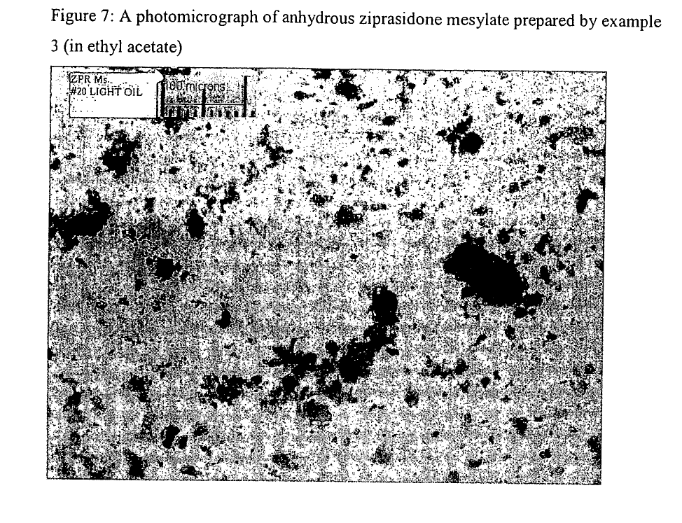

The present invention provides anhydrous ziprasidone mesylate having a tabular and equant morphology. These morphologies may be characterized by an electronic microscope, substantially as depicted in FIGS. 1-7.

The present invention further provides non hygroscopic anhydrous ziprasidone mesylate. Anhydrous ziprasidone mesylate is tested for water uptake after exposure to 80% relative humidity, at room temperature for 7 days, as summarized in the following table:

Water content

| % RH | water Content (By KF) | Crystal form (by XRD) |
|---|---|---|
| Initial | 0.5 | Anhydrous |
| 80 | 0.6 | Anhydrous |

Furthermore, the present invention provides a non-hygroscopic anhydrous ziprasidone mesylate having a tabular and equant morphology.

The present invention also provides anhydrous ziprasidone mesylate with a tabular and equant morphology, having a low residual solvent content. Preferably, the residual solvent content is less than about 0.8%, more preferably less than about 0.5%. More preferably, the residual solvent content is less than about 0.1, particularly less than 0.05 and more particularly less than 0.01%. More preferably, the residual solvent content is about 3500 to about 5000 ppm, most preferably about 4000 ppm. Preferably, the residual solvent is a C₃ to C₅ alcohol (preferably a C₃ to C₄ alcohol), C₁ to C₄ alkyl acetate or C₂ to C₆ ketone, more preferably isopropyl alcohol, isobutyl acetate or methyl isobutyl ketone.

Moreover, the present invention encompasses a new process for the preparation of anhydrous ziprasidone mesylate having a tabular and equant morphology, comprising desolvation of solvated or hydrated crystalline forms of ziprasidone mesylate, by providing a slurry of ziprasidone mesylate in a polar solvent, at a temperature above ambient (preferably about 40° C. to about 70° C., more preferably about 40° C. to about 60° C., most preferably about 60° C.). The solvent may be protic or aprotic. Preferably, the solvent is selected from the group consisting of a C₁-C₄ alcohol (preferably a C₂ to C₃ alcohol), a C₂ to C₈ ketone (preferably a C₂ to C₆ ketone) and a C₂ to C₈ alkyl ester (preferably a C₄ to C₆ alkyl ester). More preferably, the solvent is selected from the group consisting of iso-propanol, acetone, methyl isobutyl ketone and isobutyl acetate. The slurry is then stirred at a temperature ranging from an ambient temperature to reflux temperature (preferably at a temperature of from about 15° C. to about 140° C., more preferably at a temperature of from about 30° C. to about 90° C.). An especially preferred temperature range is from about 40° C. to about 70° C. The slurry is preferably stirred for about 1 to 18 hours (preferably from about 5 to about 18 hours, more preferably about 16 to about 18 hours), to induce the transformation of the solvated or hydrated forms to anhydrous ziprasidone mesylate having a tabular and equant morphology.

The crystalline forms of ziprasidone mesylate used for the above process may be Form I, Form II, Form III, Form V, Form XIII or Form XIX, described in co-pending U.S. application filed on Feb. 13, 2006 as Attorney Docket No. 1662/86506 (Application No. Awaited), and are characterized by X-ray powder diffractions as follows:

Form I is characterized by X-ray powder diffraction peaks at about 11.7, 17.3, 23.5, 24.2, and 25.2±0.2 degrees two-theta.

Form II is characterized by X-ray powder diffraction peaks at about 17.1, 18.8, 21.0, and, 23.7±0.2 degrees two-theta.

Form III is characterized by X-ray powder diffraction peaks at about 12.2, 20.9, 21.3, 24.0, 24.5±0.2 degrees two-theta.

Form V is characterized by X-ray powder diffraction peaks at about 22.1, 25.5, 26.8, 27.1 and 27.5±0.2 degrees two-theta.

Form XIII is characterized by X-ray powder diffraction peaks at about 17.1, 18.9, 20.9, 22.0, 23.6 and 24.6±0.2 degrees two-theta.

Form XIX is characterized by X-ray powder diffraction peaks at about 18.5, 22.0, 23.8, 24.2 and 26.1±0.2 degrees two-theta.

Preferably, the slurry is heated to a temperature of about 35° C. to about 70° C., more preferably about 40° C. to about 50° C.

Preferably, the slurry is heated for about 0.5 to about 5 hours, more preferably for about 1 to about 3 hours.

Preferably, about 0.2 to about 15 mL of the solvent are used, more preferably about 8 mL of the solvent are used, even more preferably about 5 to about 8 mL, and most preferably about 8 mL, per gram of the ziprasidone mesylate.

Recovery of said anhydrous ziprasidone mesylate form may be carried out by conventional techniques, preferably filtration. In order to increase yield during recovery, the slurry may be cooled before filtration to “precipitate” the crystalline form. The term precipitation in this instance is used in regard to settling down of solids in the slurry, rather than crystallization from a solution. Nevertheless, a slight amount of crystallization may occur during the cooling period if the solvent is somewhat soluble for ziprasidone mesylate.

Alternatively, the solid may be filtered at the temperature at which the slurry has been performed.

When a slurry of ziprasidone mesylate Forms I or XIX in isobutyl acetate or a slurry of ziprasidone mesylate Form I in methyl isobutyl ketone are used, the obtained anhydrous ziprasidone mesylate has a low residual solvent content. Preferably, the residual solvent content is less than about 0.8%, more preferably less than about 0.5%. More preferably, the residual solvent content is less than about 0.1, particularly less than 0.05 and more particularly less than 0.01%. More preferably, the residual solvent content is about 3500 to about 5000 ppm, most preferably about 4000 ppm.

Also provided by this invention is anhydrous ziprasidone mesylate having a tabular and equant morphology, prepared by the desolvation of solvated or hydrated crystalline forms of ziprasidone mesylate, as described above.

The present invention further encompasses a process for preparing anhydrous ziprasidone mesylate having a tabular and equant morphology, comprising heating a form of ziprasidone mesylate. The form may be any one of: Form I, Form II, Form III, Form IV, Form V, Form VI, Form VII, Form VIII, Form IX, From X, Form XIII or amorphous form, and the heating is for a time sufficient to obtain anhydrous ziprasidone mesylate having a tabular and equant morphology. Preferably the starting material is ziprasidone mesylate Form I, Form II, Form III, Form IV, Form VI, Form VIII, Form IX, Form X, Form XIII or amorphous form. Preferably, the ziprasidone mesylate forms are heated to a temperature above about 80° C., more preferably at least about 100° C., and most preferably at least about 130° C. Preferably, the ziprasidone mesylate starting material is heated at a temperature of from about 100° C. to about 220° C., more preferably from about 120° C. to about 190° C., and most preferably from about 140° C. to about 160° C. Preferably the ziprasidone starting material is heated for about 10 minutes to about 60 minutes, more preferably about 20 minutes to about 40 minutes, and most preferably for about 30 minutes.

The crystalline forms of ziprasidone mesylate are described in co-pending U.S. application filed on Feb. 13, 2006 as Attorney Docket No. 1662/86506 (Application No. Awaited), and are characterized by X-ray powder diffractions as follows:

Form IV is characterized by X-ray powder diffraction peaks at 17.1, 18.9, 22.7, 23.6, and 24.3±0.2 degrees two-theta.

Form VI is characterized by X-ray powder diffraction peaks at 15.1, 23.0, 23.5, and 23.8±0.2 degrees two-theta.

Form VII is characterized by X-ray powder diffraction peaks at 17.2, 19.0, 21.0, 24.3, and 24.9±0.2 degrees two-theta.

Form VIII is characterized by X-ray powder diffraction peaks at 17.1, 18.7, 23.8, and 24.4±0.2 degrees two-theta.

Form IX is characterized by X-ray powder diffraction peaks at 17.1, 18.7, 20.9, 23.8 and 24.3±0.2 degrees two-theta.

Form X is characterized by X-ray powder diffraction peaks at 7.8, 15.6, 17.9, 20.0 and 23.8±0.2 degrees two-theta.

The “amorphous” form used in the above process preferably contains less than about 10% crystalline forms, more preferably contains less than about 5% crystalline forms, and most preferably less than about 1% crystalline forms by weight.

Also provided by this invention is anhydrous ziprasidone mesylate having a tabular and equant morphology, prepared by the heating of the ziprasidone mesylate forms, as described above.

The present invention further encompasses a process for preparing anhydrous ziprasidone mesylate having a tabular and equant morphology, comprising combining ziprasidone base with methane-sulfonic acid in the presence of a solvent selected from the group consisting of a C₂ to C₈ ketone, a C₃-C₄ alcohol (preferably the alcohol is other than isopropanol), a C₂ to C₈ alkyl ester, an amide such as dimethylformamide, a C₁ to C₄ chlorinated alkane (preferably dichloromethane), a C₂ to C₈ ether, acetonitrile and 2-ethoxy ethanol, to obtain a reaction mixture. The reaction mixture is then stirred for a time (preferably for about 30 minutes to about 4 hours, more preferably from about 1 hour to about 2 hours) sufficient to obtain anhydrous ziprasidone mesylate having a tabular and equant morphology.

The solvent may contain water, preferably less than about 5%. Preferably, the solvent is dry, and the reaction conditions are kept anhydrous and protected from external humidity.

Preferably the organic solvent is selected from the group consisting of methyl-ethyl-ketone, acetone, iso-propanol (IPA), butyl lactate, di-ethyl-carbonate, methyl-iso-butyl-ketone, N,N-dimethyl acetamide, dimethyl formamide, dichloromethane, 2-ethoxy-ethanol, n-propanol, n-butanol, ethyl acetate, tetrahydrofuran (THF), and acetonitrile. When IPA is used, it is either dry or contains water at a ratio of about 99:1 to about 98:2 (IPA:water). Most preferably, the solvent is dry IPA, containing less than 0.3% water.

The mixture is heated to a temperature of about 2° C. to about 60° C., preferably about 25° C. to about 60° C. Preferably, the methane-sulfonic acid is added during a period of about 2 hours. The mixture is then stirred at that temperature for about 1 to about 16 hours until the transformation of the base to the mesylate salt is completed. Recovery of the crystalline is carried out by methods known in the art, such as washing, filtering and drying. Preferably, the drying is performed under vacuum at a temperature of about 65° C. The obtained anhydrous ziprasidone mesylate preferably has a low residual solvent content.

The present invention further provides a process for preparing anhydrous ziprasidone mesylate having low residual solvent content comprising adding methanesulfonic acid to a slurry of ziprasidone base in dry isopropanol, to obtain a reaction mixture, and stirring the reaction mixture to obtain anhydrous ziprasidone mesylate. It is preferred that the dry isopropanol contains less than about 5%, preferably less than about 1%, more preferably less than about 0.03% by volume of water.

In this process, the methanesulfonic acid is preferably added slowly to the mixture, i.e. added over a period of at least 1 hour, preferably over a period of at least 2 hours. The mixture may be stirred at a temperature of about 20° C. to about 70° C., preferably about 60° C. to about 70° C.

Also provided by this invention is anhydrous ziprasidone mesylate having a tabular and equant morphology, prepared by the process described above.

The present invention further encompasses a process for preparing anhydrous ziprasidone mesylate having a tabular and equant morphology, comprising combining ziprasidone base with methane-sulfonic acid in the presence of a mixture of THF and water or 1,4-dioxane, to obtain a reaction mixture. The reaction mixture is then stirred at a temperature of about 25° C. to about 50° C. for a time sufficient to obtain anhydrous ziprasidone mesylate having a tabular and equant morphology.

Also provided by this invention is anhydrous ziprasidone mesylate having a tabular and equant morphology, prepared by the process described above.

The present invention related to pharmaceutical formulations comprising anhydrous ziprasidone mesylate having a tabular and equant morphology and a pharmaceutically acceptable carrier.

The present invention is also related to pharmaceutical formulations comprising anhydrous ziprasidone mesylate with a tabular and equant morphology, having a low residual solvent content, and a pharmaceutically acceptable carrier.

The present invention provides the use of ziprasidone mesylate having a tabular and equant morphology for the preparation of solid pharmaceutical formulation.

The present invention further provides the use of non-hygroscopic anhydrous ziprasidone mesylate for the preparation of solid pharmaceutical formulation.

The present invention also provides a solid pharmaceutical formulation containing a lyophilizate of anhydrous ziprasidone mesylate that has a tabular and equant morphology.

The present invention further provides the preparation of pharmaceutical formulations containing ziprasidone mesylate by lyophilizing anhydrous ziprasidone mesylate having a tabular and equant morphology, or by lyophilizing non-hygroscopic anhydrous ziprasidone mesylate.

Lyophilization is carried on by procedures well known in the art, which include cooling a water solution of the active ingredient (preferably mixed with excipients, optionally excipients that may increase the solubility of the active ingredient). After all the solution freezes (in pure ice crystals and eutectic mixture of water/solute), vacuum drying is applied until all the water has sublimed off. The remaining cake can be dried at different water levels, typically up to 1-2% water content (J. T. Carstensen, Pharmaceutical principles of solid dosage forms, 1993, Technomic Publishing Company).

The present invention provides a solid pharmaceutical formulation containing nano-particles (e.g. having an average particle size of less than about 100 nm, more preferably less than about 50 nm, even more preferably less than about 10 nm, and most preferably less than about 5 nm) produced using anhydrous ziprasidone mesylate having a tabular and equant morphology.

The present invention also provides a process for the preparation of this pharmaceutical formulation by dissolving said ziprasidone mesylate in a mixture of N-methyl-pyrrolidone and water, and adding pharmaceutical ingredients to the solution (such as sorbitol). Subsequently, cooling the solution to about −40° C. and lyophilizing to give a powder containing nano-particles of ziprasidone Mesylate.

As used herein, the term “pharmaceutical formulations” includes tablets, pills, powders, liquids, suspensions, emulsions, granules, capsules, suppositories, or injection preparations. Preferably, the pharmaceutical formulations according to the present invention are solid formulations, such as tablets, capsules, powders, suspensions, granules and suppositories. Pharmaceutical formulations containing the ziprasidone mesylate of the invention may be prepared by using diluents or excipients such as fillers, bulking agents, binders, wetting agents, disintegrating agents, surface active agents, and lubricants. Various modes of administration of the pharmaceutical formulations of the invention can be selected depending on the therapeutic purpose, for example tablets, pills, powders, liquids, suspensions, emulsions, granules, capsules, suppositories, or injection preparations.

Any excipient commonly known and used widely in the art can be used in the pharmaceutical formulation. Carriers used include, but are not limited to, lactose, white sugar, sodium chloride, glucose, urea, starch, calcium carbonate, kaolin, crystalline cellulose, silicic acid, and the like. Binders used include, but are not limited to, water, ethanol, propanol, simple syrup, glucose solutions, starch solutions, gelatin solutions, carboxymethyl cellulose, shellac, methyl cellulose, potassium phosphate, polyvinylpyrrolidone, and the like. Disintegrating agents used include, but are not limited to, dried starch, sodium alginate, agar powder, laminalia powder, sodium hydrogen carbonate, calcium carbonate, fatty acid esters of polyoxyethylene sorbitan, sodium laurylsulfate, monoglyceride of stearic acid, starch, lactose, and the like. Disintegration inhibitors used include, but are not limited to, white sugar, stearin, coconut butter, hydrogenated oils, and the like. Absorption accelerators used include, but are not limited to, quaternary ammonium base, sodium laurylsulfate, and the like. Wetting agents used include, but are not limited to, glycerin, starch, and the like. Adsorbing agents used include, but are not limited to, starch, lactose, kaolin, bentonite, colloidal silicic acid, and the like. Lubricants used include, but are not limited to, purified talc, stearates, boric acid powder, polyethylene glycol, and the like. Tablets can be further coated with commonly known coating materials such as sugar coated tablets, gelatin film coated tablets, tablets coated with enteric coatings, tablets coated with films, double layered tablets, and multi-layered tablets.

When shaping the pharmaceutical formulation into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical formulation in the form of suppositories, any commonly known excipient used in the art can be used. For example, excipients include, but are not limited to, polyethylene glycols, coconut butter, higher alcohols, esters of higher alcohols, gelatin, and semisynthesized glycerides.

When preparing injectable pharmaceutical formulations, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic.

Additional ingredients, such as dissolving agents, buffer agents, and analgesic agents may be added. If necessary, coloring agents, preservatives, perfumes, seasoning agents, sweetening agents, and other medicines may also be added to the desired preparations.

The amount of the ziprasidone mesylate of the present invention contained in a pharmaceutical formulation for treating schizophrenia should be sufficient to treat, ameliorate, or reduce the symptoms associated with schizophrenia. Preferably, the ziprasidone mesylate of the present invention is present in an amount of about 1% to about 70% by weight, and more preferably from about 1% to about 30% by weight of the dose.

The pharmaceutical formulations of the invention may be administered in a variety of methods depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations may be administered intramuscularly, intracutaneously, subcutaneously or intraperitoneally. Suppositories may be administered into the rectum.

The dosage of a pharmaceutical formulation for treating schizophrenia according to the invention will depend on the method of use, the age, sex, and condition of the patient. Preferably, the ziprasidone mesylate of the present invention is administered in an amount from about 0.1 mg/kg to about 10 mg/kg of body weight/day. More preferably, about 1 mg to 200 mg of ziprasidone mesylate may be contained in a dose.

Having described the invention with reference to certain preferred embodiments, other embodiments will become apparent to one skilled in the art from consideration of the specification. The invention is further defined by reference to the following examples describing in detail the analysis of the anhydrous ziprasidone mesylate and methods for preparing thereof.

It will be apparent to those skilled in the art that many modifications, both to materials and methods, may be practiced without departing from the scope of the invention.

### EXAMPLES

Experimental

X-Ray powder diffraction data were obtained using a SCINTAG powder X-Ray diffractometer model X'TRA equipped with a solid state detector. Copper radiation of 1.5418 Å was used. A round aluminum sample holder with zero background was used. All peak positions are within ±0.2 degrees two theta. Karl Fisher analysis was performed according to the known art.

Microscope: The material was dispersed in a light mineral oil before the measurement.

The residual solvents were measured y GC-head space using GC HS Agilent-Technology 6890.

Unless otherwise stated, the drying of the products are carried out at a temperature of about 50° C., at about 20 mmHg for a period of about 16 hours.

### Example 1

### Preparation of Ziprasidone Mesylate Anhydrous with Tabular and Equant Morphology from Form X

The wet solid Ziprasidone mesylate form X obtained from the mixture of THF and water in a ratio of 99:1 at 5° C., was dried in a hood. After drying the solid was analyzed by XRD giving Ziprasidone mesylate anhydrous.

### Example 2

### Preparation of Ziprasidone Mesylate Anhydrous with Tabular and Equant Morphology from Form XIII

To slurry of Ziprasidone base (3 g) in absolute ethanol (AR) (30 ml) at about 50° C., was added methane sulfonic (0.6 ml). Then, the mechanically stirred slurry was heated to 65° C. for 30 min. The reaction mixture was then cooled to room temperature and further stirred for one hour. The solid was then filtered, washed with ethanol and dried (in oven vacuum at 45° C. and in hood). The obtained wet and dried solids, ziprasidone mesylate form XIII (water content by K.F. 0.3%), were heated in IPA at 50° C., and then filtered and dried giving zipasidone mesylate anhydrous.

### Example 3

### Preparation of Ziprasidone Mesylate Anhydrous having Equant and Tabular Morphology

To the solution or slurry of Ziprasidone base in an organic polar solvent (protic and aprotic solvent) (10 mL per gram of ziprasidone base) was added methane-sulfonic acid (1.1 equivalents), at a temperature in the range of 2-60° C. The obtained slurry was then stirred for 30 min. to 2 hours, followed by filtering the solid, washing with the solvent and drying, usually in a vacuum oven at about 50-60° C. The dried solid was Ziprasidone mesylate anhydrous, with a morphology of tabular and equant.

The representative examples are summarized in following table:

| | | Crystal form | Morphology (by |
|---|---|---|---|
| Solvent | Temperature | (by XRD) | microscope) |
| Methyl-ethyl-ketone | 25° C. | anhydrous | Tabular and equant |
| acetone | 50° C. | anhydrous | Tabular and equant |
| THF/water 99:1 | 25-50° C. | anhydrous | Tabular and equant |
| Butyl acetate | 25-50° C. | anhydrous | Tabular and equant |
| Butyl lactate | 25-50° C. | anhydrous | Tabular and equant |
| di-Ethyl-carbonate | 25-50° C. | anhydrous | Tabular and equant |
| Methyl-iso-butyl- | 25-50° C. | anhydrous | Tabular and equant |
| ketone | | | |
| 1,4-Dioxane | 25-50° C. | anhydrous | Tabular and equant |
| N,N-dimethyl- | 25-50° C. | anhydrous | Tabular and equant |
| acetamide | | | |
| Dimethyl-formamide | 25-50° C. | anhydrous | Tabular and equant |
| Dichloro-methane | 25-50° C. | anhydrous | Tabular and equant |
| 2-ethoxy-ethanol | 50° C. | anhydrous | Tabular and equant |
| n-propanol | 50° C. | anhydrous | Tabular and equant |
| n-butanol | 50° C. | anhydrous | Tabular and equant |
| Ethyl acetate | 50° C. | anhydrous | Tabular and equant |
| tetrahydrofuran | ˜5° C. | anhydrous | Tabular and equant |
| acetonitrile | 50° C. | anhydrous | Tabular and equant |

### Example 4

### Preparation of Ziprasidone Mesylate Anhydrous, having Tabular and Equant Morphology, by Desolvation of a Solvated/Hydrated Form

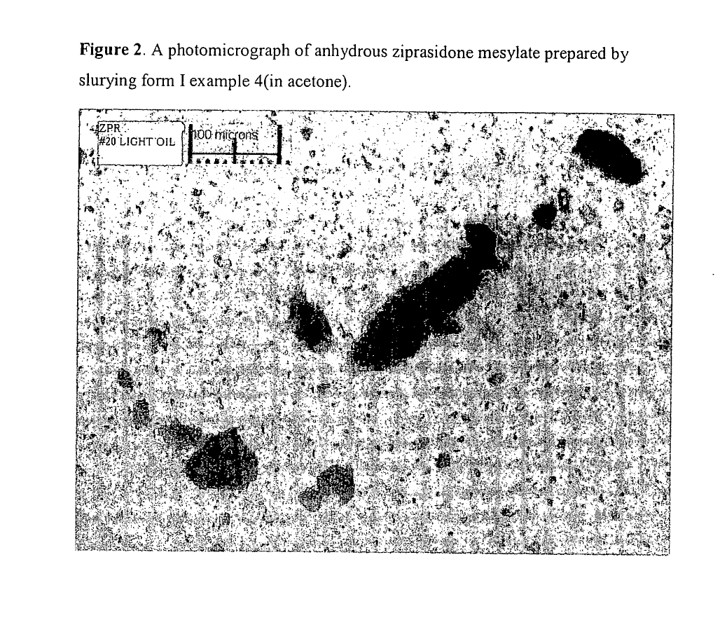

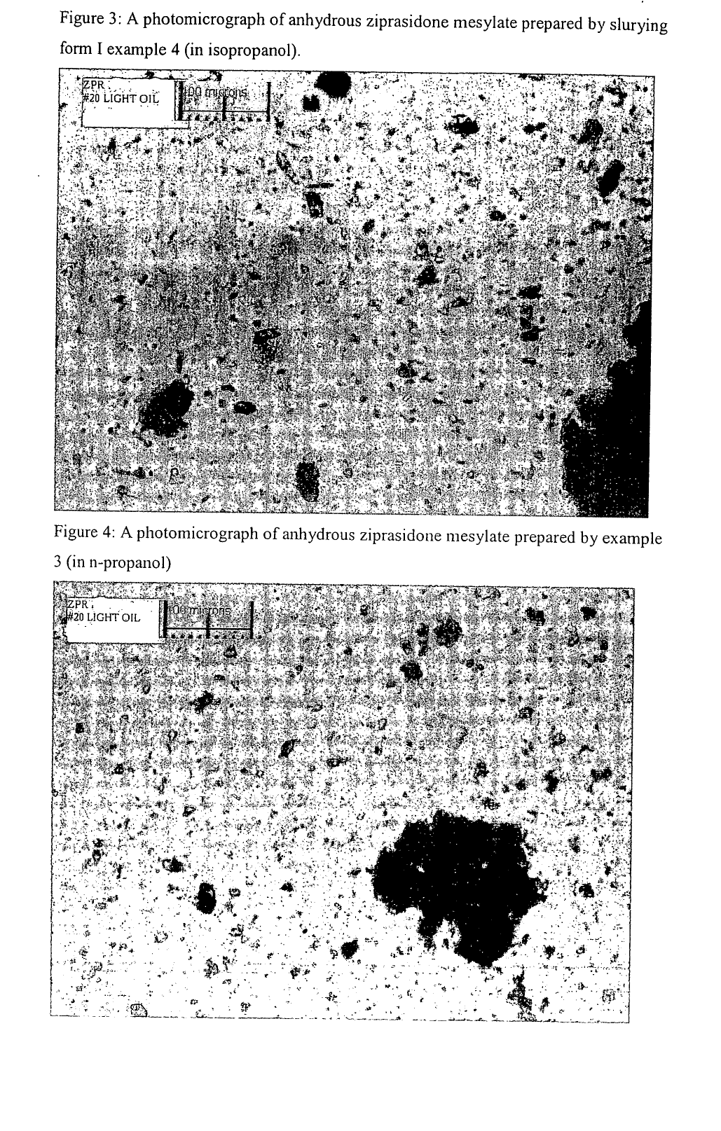

The slurry of Ziprasidone mesylate (solvated/hydrated crystalline form) in a polar solvent (protic and aprotic) (10-20 mL per gram of ziprasidone mesylate) is stirred at a temperature in the range of ambient temperature to reflux temperature, for a time sufficient to induce the transformation of form I to form anhydrous, having the morphology of the crystalline form of the present invention.

| | XRD of | | | | |
|---|---|---|---|---|---|
| | starting | | | Crystal form | Morphology (by |
| Solvent | material | Temp. | Time | (by XRD) | microscope) |
| acetone | I | 40° C. | 3 h | anhydrous | Tabular and |
| | | | | | equant (FIG. 2 |
| Iso-propanol | I | 40° C. | 3 h | anhydrous | Tabular and |
| | | | | | equant (FIG. 3 |
| Iso-propanol | I | 50° C. | 2 h | anhydrous | Tabular and |
| | | | | | equant |
| Iso-propanol | II | 40° C. | 1 h | anhydrous | Tabular and |
| | | | | | equant |
| acetone | II | 50° C. | 2 h | anhydrous | Tabular and |
| | | | | | equant |
| Iso-propanol | III | 40° C. | 1 h | anhydrous | Tabular and |
| | | | | | equant |
| Iso-propanol | V | 40° C. | 1 h | anhydrous | Tabular and |
| | | | | | equant |
| Iso-propanol | XIII | 40° C. | 1 h | anhydrous | Tabular and |
| | | | | | equant |

### Example 5

### Preparation of Ziprasidone Mesylate Anhydrous, having Tabular and Equant Morphology, by Heating Ziprasidone Mesylate Polymorphs

In the following examples, ziprasidone mesylate forms were heated in an oven at the specified temperature and time periods indicated in the following table.

TABLE 1
TRANSFORMATIONS BY HEAT

| Starting | Temp | Time | | |
|---|---|---|---|---|
| material | [° C.] | [min] | Obtained form | Morphology |
| Form I | 160 | 30 | Anhydrous | Equant and tabular |
| Form I + | 130 | 30 | Anhydrous | Equant and tabular |
| Amorphous | | | | |
| Form III | 160 | 30 | Anhydrous | Equant and tabular |
| Form II | 160 | 30 | Anhydrous | Equant and tabular |
| Form VI | 160 | 30 | Anhydrous | Equant and tabular |
| Form V | 120 | 30 | Anhydrous | Lath, equant and tabular |
| Form V | 160 | 30 | Anhydrous | Lath, equant and tabular |
| Form VII | 160 | 30 | Anhydrous | Lath, equant and tabular |
| Form IV | 160 | 30 | Anhydrous | Equant and tabular |
| Form VIII | 160 | 30 | Anhydrous | Equant and tabular |
| Amorphous | 190 | 30 | Anhydrous | Equant and tabular |
| Form IX | 140 | 30 | Anhydrous | Equant and tabular |
| Form X | 140 | 30 | Anhydrous | Equant and tabular |
| Form XIII | 140 | 30 | Anhydrous | Equant and tabular |

### Example 6

### Preparation of Ziprasidone Mesylate Anhydrous having a Low Residual Solvent Content, from IPA

To Ziprasidone base (20 g) was added isopropyl alcohol (IPA) extra dry (containing less than about 0.03% water) (320 ml) and the obtained slurry was heated under nitrogen to about 60° C. Methanesulfonic acid (3.44 ml) in extra dry isopropyl alcohol (80 ml) was slowly added during 2 hours. The mixture was than stirred at that temperature for about 16 hours. The solid was filtrated under nitrogen atmosphere, washed with extra dry isopropyl alcohol (2×20 ml) and dried in a vacuum oven at 65° C. and 7 mm Hg for about 16 hours. The dried material was Ziprasidone mesylate Anhydrous having a residual solvent IPA of 4613 ppm (by GC head space).

### Example 7

### Preparation of Ziprasidone Mesylate Anhydrous with Tabular and Equant Morphology, having a Low Residual Solvent Content, Via Form I

a) Preparation of Ziprasidone Mesylate form I

Ziprasidone base (50 g) was dissolved in a mixture of acetic acid (312.5 ml) and isobutylacetate (i-BuOAc) (187.5 ml) and the solution was treated with active carbon and tonsil at 0-5° C. for color improvement. The solution was filtrated and gently heated to the room temperature. Methanesulfonic acid was added (8.26 ml) keeping the temperature in the range 20-25° C. After about 10 min. stirring precipitation starts; the stirring was maintained for additional 2.5 hours. After this the solid was filtrated, washed with i-BuOAc (50 ml). A small sample was dried for full characterization. The obtained material was Ziprasidone mesylate form I. The wet material was taken for the subsequent slurry operation.

b) Preparation of Ziprasidone Mesylate Anhydrous by Slurry of Form I in i-BuOAc

To the wet material Ziprasidone mesylate form I (4 g) obtained as above i-BuOAc was added (20 ml) and the slurry was heated at 60° C. for 16 hours. The solid was filtrated at that temperature, washed with i-BuOAc (5 ml) and dried in vacuum-oven at 65° C. The dried solid was Ziprasidone mesylate Anhydrous (Residual solvents: GC head space: i-BuOAc 1774 ppm, i-BuOH 50 ppm; HPLC: acetic acid 1191 ppm Total=3015 ppm).

### Example 8

### Preparation of Ziprasidone Mesylate Anhydrous with Tabular and Equant Morphology, having a Low Residual Solvent Content, Via form I

a) Preparation of Ziprasidone Mesylate Form I

Ziprasidone base (30 g) was dissolved in a mixture of acetic acid (187.5 ml) and methylisobutylketone (MIKB) (187.5 ml) and the solution was treated with active carbon and tonsil at 0-2° C. for color improvement. The solution was filtrated and gently heated to the room temperature. Methanesulfonic acid was added (4.92 ml) keeping the temperature in the range 20-22° C. During the addition precipitation starts; the stirring was maintained for additional 3 hours. After this the solid was filtrated under nitrogen atmosphere, washed with MIBK (40 ml). A small sample was dried for full characterization. The obtained material was Ziprasidone mesylate form I. The wet material was taken for the subsequent slurry operation.

b) Preparation of Ziprasidone Mesylate Anhydrous by Slurry of Form I in MIBK

To the wet material Ziprasidone mesylate form I (10 g) obtained as above, MIBK was added (150 ml) and the slurry was heated at 50-60° C. for 3 hours. The solid was filtrated at that temperature, washed with MIBK (5 ml) and dried in vacuum-oven at 65° C. The dried solid was Ziprasidone mesylate Anhydrous (Residual solvents: GC head space: MIBK 3024 ppm, HPLC: acetic acid 807 ppm. Total=3831 ppm).

### Example 9

### Preparation of Ziprasidone Mesylate Anhydrous having a Low Residual Solvent Content from Form XIX:

The slurry of Ziprasidone mesylate wet form XIX (5 g) in i-BuOAc (25 ml) was heated under nitrogen atmosphere at 60° C. and the stirring at that temperature was applied for 16 hours. After this the solid was filtrated from the hot slurry, washed with hot i-BuOAc and dried in oven at 65° C. for about 16 hours. The dried material was Ziprasidone mesylate Anhydrous (residual solvent: i-BuOAc (by GC-HS) 2196 ppm, Acetic acid (by HPLC) 1246 ppm; total 3532 ppm).

### Example 10

### Testing Absorption of Water of Ziprasidone Mesylate Anhydrous, having Equant and Tabular Morphology

Anhydrous ziprasidone mesylate, having a tabular, and equant morphology was exposed to 80% humidity for at least 7 days at room temperature. Before the exposure, the water content was 0.5% as measured by Karl Fisher. After 7 days at 80% humidity at RT the sample contained 0.6% water as measured by Karl Fisher. Thus, there was an increase of 0.1% in the water content. No change in the crystal form was found after the exposure.

### Example 11

### Procedure for the Preparation of Nano-particles Ziprasidone Mesylate

Ziprasidone Mesylate anhydrous having a tabular and equant morphology is dissolved in a mixture of N-methyl-pyrrolidone and water. To this solution pharmaceutical ingredients (such as sorbitol) are added. The solution is cooled to about −40° C. and lyophilized to give a powder containing nano-particles of Ziprasidone Mesylate.

We claim:

1. Anhydrous ziprasidone mesylate having a tabular and equant morphology.

2. The anhydrous ziprasidone mesylate of claim 1, characterized by an electronic microscope, substantially as depicted in FIGS. 1 to 7.

3. The anhydrous ziprasidone mesylate of claim 1 or claim 2, which is non-hygroscopic.

4. Non hygroscopic anhydrous ziprasidone mesylate.

5. The anhydrous ziprasidone mesylate of claim any preceding claim, having a low residual solvent content.

6. The anhydrous ziprasidone mesylate of claim 5, wherein the residual solvent is a C₃ to C₅ alcohol, C₁ to C₄ alkyl acetate or C₂ to C₆ ketone.

7. The anhydrous ziprasidone mesylate of claim 6, wherein the residual solvent is isopropyl alcohol, isobutyl acetate or methyl isobutyl ketone.

8. A process for the preparation of the anhydrous ziprasidone mesylate of any preceding claim, comprising desolvation of solvated or hydrated crystalline forms of ziprasidone mesylate.

9. The process of claim 8, wherein the desolvation process is performed by providing a slurry of ziprasidone mesylate in a polar solvent; stirring the slurry at a temperature ranging from an ambient temperature to reflux temperature, for about 1 to 18 hours, to induce the transformation of the solvated or hydrated forms to anhydrous ziprasidone mesylate having a tabular and equant morphology; and recovering anhydrous ziprasidone mesylate.

10. The process of claim 9, wherein the polar solvent is selected from the group consisting of a C₁-C₄ alcohol, C₂ to C₈ ketone and a C₂ to C₈ alkyl ester.

11. The process of claim 10, wherein the polar solvent is selected from the group consisting of iso-propanol, acetone, methyl isobutyl ketone and isobutyl acetate.

12. The process of any of claims 9 to 11, wherein the slurry is stirred at a temperature of about 40° C. to about 70° C.

13. The process of claim 12, wherein the slurry is stirred at a temperature of about 40° C. to about 60° C.

14. The process of any of claims 9 to 13, wherein the slurry is stirred for about 5 to about 18 hours.

15. The process of claim 14, wherein, the slurry is stirred for about 16 to about 18 hours.

16. The process of any of claims 9 to 15, wherein the slurry provided contains a crystalline form of ziprasidone mesylate selected from the group consisting of:
Form I, Form II, Form III, Form V, Form XIII and Form XIX.

17. The process of claim 16, wherein when a slurry of ziprasidone mesylate Forms I or XIX in isobutyl acetate or a slurry of ziprasidone mesylate Form I in methyl isobutyl ketone are used, the obtained anhydrous ziprasidone mesylate has a low residual solvent content.

18. The process of any of claims 9 to 17, wherein about 0.2 mL to about 15 mL of the solvent are used, per gram of the ziprasidone mesylate.

19. The process of claim 18 wherein about 0.2 to about 8 mL of the solvent are used, per gram of the ziprasidone mesylate.

20. The process of claim 19, wherein about 5 to about 8 mL are used, per gram of the ziprasidone mesylate.

21. The process of claim 20, wherein about 8 mL are used, per gram of the ziprasidone mesylate.

22. A process for preparing the anhydrous ziprasidone mesylate of claim 1, comprising heating ziprasidone mesylate Form I, Form II, Form III, Form IV, Form V, Form VI, Form VII, Form VIII, Form IX, From X, Form XIII or amorphous form to a temperature above about 80° C.

23. The process in claim 22, wherein the anhydrous ziprasidone mesylate is prepared by heating ziprasidone mesylate crystalline Form I, Form II, Form III, Form IV, Form VI, Form VIII, Form IX, Form X, Form XIII or amorphous form to a temperature above about 80° C.

24. The process of claim 22 or claim 23, wherein the ziprasidone mesylate form is heated to a temperature above about 80° C., preferably above about 100° C.

25. The process of claim 24, wherein the ziprasidone mesylate form is heated to a temperature of at least about 120° C.

26. The process of claim 25, wherein the ziprasidone mesylate form is heated to a temperature of at least about 130° C.

27. A process for preparing the anhydrous ziprasidone mesylate according to any of claims 1 to 8, comprising combining ziprasidone base with methane-sulfonic acid in the presence of a solvent selected from the group consisting of: a C₂ to C₈ ketone, a C₃-C₄ alcohol other than isopropanol, a C₂ to C₈ alkyl ester, an amide, a chlorinated C₁ to C₄ alkane, a C2 to C₈ ether, acetonitrile, 2-ethoxy ethanol, dioxane and a mixture of THF and water, to obtain a reaction mixture; and stirring the reaction mixture for a time sufficient to obtain the anhydrous ziprasidone mesylate.

28. The process of claim 27, wherein the organic solvent is selected from the group consisting of methyl-ethyl-ketone, acetone, butyl lactate, di-ethyl-carbonate, methyl-iso-butyl-ketone, N,N-dimethyl acetamide, dimethyl formamide, dichloromethane, 2-ethoxy-ethanol, n-propanol, n-butanol, ethyl acetate, tetrahydrofuran, and acetonitrile.

29. The process of claim 27 or claim 28, wherein the organic solvent contains less than about 5% water by volume.

30. The process of any of claims 27 to claim 29, wherein the reaction mixture is heated to a temperature of about 2° C. to about 60° C.

31. The process of claim 30, wherein the reaction mixture is heated to a temperature of about 25° C. to about 60° C.

32. The process of claim 31 wherein the reaction mixture is heated to a temperature of about 25° C to about 50° C.

33. The process of any of claims 27 to 32, wherein the methane-sulfonic acid is added during a period of about 2 hours.

34. The process of claim 33, wherein the reaction mixture is stirred for about 1 to about 16 hours, preferably for about 30 min to 2 hours.

35. A process for preparing anhydrous ziprasidone mesylate having low residual solvent content comprising adding methanesulfonic acid to a slurry of ziprasidone base in dry isopropanol, to obtain a reaction mixture, and stirring the reaction mixture to obtain anhydrous ziprasidone mesylate.

36. The process of claim 35 wherein the isopropanol has a water content of less than about 5% by volume.

37. The process of claim 36 wherein the isopropanol has a water content of less than about 1% by volume.

38. The process of claim 37 wherein the isopropanol has a water content of less than about 0.03% by volume.

39. The process of any of claims 35 to 38 wherein the methanesulfonic acid is added over a period of at least 1 hour.

40. The process of claim 39 wherein the methanesulfonic acid is added over a period of at least 2 hours.

41. The process of any of claims 35 to 40 wherein the mixture is stirred at a temperature of about 20° C. to about 70° C.

42. The process of claim 41 wherein the mixture is stirred at a temperature of about 60° C. to about 70° C.

43. A pharmaceutical formulation comprising the anhydrous ziprasidone mesylate of any one of claims 1 to 7, and a pharmaceutically acceptable carrier.

44. A solid pharmaceutical formulation as defined in claim 43.

45. A pharmaceutical formulation containing a lyophilizate of the anhydrous ziprasidone mesylate of any of claims 1 to 7.

46. A pharmaceutical formulation containing nano-particles of the anhydrous ziprasidone mesylate of any of claims 1 to 7.

47. A process for the preparation of the pharmaceutical formulation of claim 45 or claim 46, comprising dissolving the anhydrous ziprasidone mesylate of any of claims 1 to 7 in a mixture of N-methyl-pyrrolidone and water, adding pharmaceutical ingredients to the solution, cooling the solution to about −40° C. and lyophilizing.

48. Use of the anhydrous ziprasidone mesylate of any of claims 1 to 7 in the manufacture of a pharmaceutical formulation.
